(19) United States
(12) Patent Application Publication
(10) Pub. No.: US 2024/0067138 A1
(43) Pub. Date: Feb. 29, 2024

(54) Apparatus with Magnetic Drive for Generation of Air-Borne Spray to Remove Malodor and Contamination

(71) Applicant: NUVINAIR, LLC, Plano, TX (US)

(72) Inventors: Kyle Bailey, Plano, TX (US); Jason Bailey, Plano, TX (US)

(21) Appl. No.: 18/386,951

(22) Filed: Nov. 3, 2023

Related U.S. Application Data
(63) Continuation of application No. 17/989,454, filed on Nov. 17, 2022.
Application No. 17/989,454 is a continuation of application No. 16/835,851, filed on Mar. 31, 2020, now Pat. No. 11,535,205.
Application No. 16/835,851 is a continuation-in-part of application No. 15/895,510, filed on Feb. 13, 2018, now Pat. No. 11,420,599.
Application No. 15/895,510 is a continuation of application No. 15/253,315, filed on Aug. 31, 2016, now Pat. No. 9,925,959.
Application No. 15/253,315 is a division of application No. 15/063,931, filed on Mar. 8, 2016, now Pat. No. 9,446,742.
Application No. 15/063,931 is a continuation-in-part of application No. 18/121,944, filed on Mar. 15, 2023.
Application No. 18/121,944 is a continuation of application No. 17/989,454, filed on Nov. 17, 2022.
Application No. 17/989,454 is a continuation of application No. 16/835,851, filed on Mar. 31, 2020, now Pat. No. 11,535,205.
Application No. 16/835,851 is a continuation-in-part of application No. 15/895,510, filed on Feb. 13, 2018, now Pat. No. 11,420,599.
Application No. 15/895,510 is a continuation of application No. 15/253,315, filed on Aug. 31, 2016, now Pat. No. 9,925,959.
Application No. 15/253,315 is a division of application No. 15/063,931, filed on Mar. 8, 2016, now Pat. No. 9,446,742.
(60) Provisional application No. 62/253,188, filed on Nov. 10, 2015.
(60) Provisional application No. 62/253,188, filed on Nov. 10, 2015.

(51) Int. Cl. B60S 3/00; A61L 2/22; A61L 9/14; B08B 3/02; B08B 3/04; B08B 9/00

(52) U.S. Cl. CPC B60S 3/008; A61L 2/22; A61L 9/14; B08B 3/02; B08B 3/04; B08B 9/00; A61L 2202/16; A61L 2202/17

(57) ABSTRACT

There is provided an apparatus, system, and method of treating a condition in an enclosable environment. The method includes adding water and a formulation into a container and closing the enclosable environment with the container there inside. The water and formulation are agitated via a magnetic impeller, thereby causing a release of a gaseous cleaning agent from the formulation. An air-borne spray exiting from the container spreads throughout the enclosable environment to contact the air and surfaces to be cleaned, thereby decontaminating, and removing malodor from within the enclosable environment. The enclosable environment is maintained closed for an effective time period while the cleaning agent treats the enclosable space.

### STATEMENT OF RELATED APPLICATIONS

This application is a continuation-in-part application of (1) pending U.S. Ser. No. 17/989,454, filed on Nov. 17, 2022, which is a continuation of U.S. Ser. No. 16/835,851, filed on Mar. 31, 2020, issued as U.S. Pat. No. 11,535,205, which is a continuation-in-part application of U.S. Ser. No. 15/895,510, filed Feb. 13, 2018, issued as U.S. Pat. No. 11,420,599, which is in turn a continuation of application U.S. Ser. No. 15/253,315, filed Aug. 31, 2016, issued as U.S. Pat. No. 9,925,959, which is in turn a divisional application of U.S. Ser. No. 15/063,931, filed on Mar. 8, 2016, and issued as U.S. Pat. No. 9,446,742, which claims the benefit of provisional application 62/253,188, filed on Nov. 10, 2015; and (2) pending U.S. Ser. No. 18/121,944, filed on Mar. 15, 2023, which is a continuation of pending U.S. Ser. No. 17/989,454, filed on Nov. 17, 2022, which is a continuation of U.S. Ser. No. 16/835,851, filed on Mar. 31, 2020, issued as U.S. Pat. No. 11,535,205, which is a continuation-in-part application of U.S. Ser. No. 15/895,510, filed Feb. 13, 2018, issued as U.S. Pat. No. 11,420,599, which is in turn a continuation of application U.S. Ser. No. 15/253,315, filed Aug. 31, 2016, issued as U.S. Pat. No. 9,925,959, which is in turn a divisional application of U.S. Ser. No. 15/063,931, filed on Mar. 8, 2016, and issued as U.S. Pat. No. 9,446,742, which claims the benefit of provisional application 62/253,188, filed on Nov. 10, 2015. The entire contents of all the foregoing applications are hereby incorporated by reference herein.

### BACKGROUND

### 1. Field of the Invention

The present technology relates to apparatus and systems for air-borne dispersal of a cleaning agent onto contaminated surfaces to clean these surfaces. More particularly, the technology relates to cleaning of surfaces, contaminated with microbial and/or a viral load that may be hazardous to human health and that may lead to contagion, in enclosed spaces, such as vehicle people-carrying spaces, to reduce or eliminate the contamination.

### 2. Description of the Related Art

There is increasing concern about the spread of contagious diseases, whether these may be influenza, common colds, corona viruses (SARS, MERS, COVID-19), or a potentially lethal virus such as Ebola, or microbial or viral diseases that are not even known or identified at this time. For purposes of this description, microscopic fungi, bacteria and viruses are included in the term “microbes.” Most of these microbes and viruses are spread through contact; a first person contacts some surface (for example, by shaking the hand of a contagious person or touches a contaminated surface) and acquires the contamination, becomes infected, and then passes it on to yet another person. This chain of infection is well-known. Some contaminants, whether microbial or viral, appear to be spread through “air-borne” means. This includes coughing and the emitting of a fine spray of contaminated and contagious sputum.

In a modern urban environment, one of the main means of transportation is enclosed vehicles such as, but not limited to, aircraft, busses, trains, boats, cars, SUVs and trucks. Some of these are vehicles that are open to the general public to use, and some members of the public may have a communicable disease that is spread through microbes or viruses. Surfaces inside the cabin of the vehicle, where passengers are usually seated, may over time become heavily contaminated with live microbes and viral contaminants. Thus, these surfaces serve to spread the microbial or viral disease to other passengers through contact.

Even in non-public, personal or family transportation, one family member may be ill and could contaminate surfaces thereby passing a contagious illness to other family members. This is especially a risk where school-aged children “pick up” a microbial or viral infection from classmates at school, and can then pass it on to parents and siblings through contaminated surfaces in a family vehicle. Some microbes or viruses may be long-lived, and immunity to these may not be readily achieved. Thus, there is a chance of recurrent illness. Merely wiping surfaces may not eliminate the microbial or viral load on surfaces because surfaces may not be smooth and totally accessible. For example, surfaces are often textured and may have joints and other features where microbial and viral loads may persist.

With regard to newly manufactured vehicles, the chances of a microbial or viral load on surfaces are low, unless the vehicle was contaminated during assembly. On the other hand, the chances that a “pre-owned” or “used” vehicle is contaminated and a source of potential infection, is relatively far higher. Aside from the potential health issues, there are often also aesthetic issues with pre-owned or used cars: they may have an odor in the cabin space from pets carried in the space or from the way in which they were (mis)used by the previous owners. This can have a negative impact on the resale value of the vehicle.

There is a need from a public health standpoint to clean surfaces within a passenger carrying cabin space of vehicles to reduce any microbial and/or viral load. Moreover, there is also not only a public health need to do this but also a business or economic need to remove any undesirable odors from the cabin space of public, used or pre-owned vehicles.

There are other spaces besides where microbial contaminations may linger as well. These include but are not limited to: rooms in a house, hotel rooms, hospital rooms, rooms in homes for the aged, intensive care units, surgery rooms, yoga rooms, gyms, restaurants, ships cabins and passenger-use spaces on cruise vessels, trains, buses, aircraft cabins, etc. In general, living spaces and other spaces that humans use regularly and that contain surfaces on which microbes can dwell, are a potential source for spread of infections.

### SUMMARY

This summary is intended to present a brief outline of some of the features of exemplary embodiments of the inventions; these and additional features are more particularly described in the Detailed Description, here below. The descriptions do not limit the scope on the inventions, which is set forth in the appended patent claims.

In an exemplary embodiment, there is provided an apparatus for generating and distributing a gaseous cleaning agent. The apparatus includes a container having an interior volume configured to contain water and a formulation that produces the gaseous cleaning agent when in contact with the water. The apparatus also includes a magnetic impeller located in the container. There is a base coupled to the lower end of the container. In addition, there is a rotating magnetic field generated in the base, the magnetic field interacting with the magnetic impeller and thereby causing the impeller to rotate along with the magnetic field. The rotation of the magnetic field is such that the magnetic impeller rotates with sufficient speed in the container, containing the water and the formulation, that a vortex forms in the container. The rotation and the vortex accelerates release of the gaseous cleaning agent, and that generates an air-borne spray of water and gaseous cleaning agent from the container.

In an exemplary embodiment, the rotating magnetic field is created by a magnet mechanically coupled to a spindle of motor, both located in the base. The magnet provides the magnetic field and rotation of the motor causes rotation of the magnet and thereby rotation of the magnetic field.

Optionally, the magnetic impeller also includes knob disposed on the midsection, which allows the magnetic impeller to rotate on the knob.

To avoid leakage of liquid from the interior of the container to the motor, and other electronics in the base, the base is isolated from the contents of the container, such that the motor in the base is not in fluid communication with the container interior but is sealed off from the container interior. In one example, the motor is in a cavity within the base, along with the magnet.

Optionally, the base includes a mounting interface configured to engage or register with a corresponding mounting interface at the lower end of the container. The mounting interfaces may engage each other by mechanical coupling, for example.

In yet another exemplary embodiment, there is provided a method of generating and distributing a gaseous cleaning agent to treat a condition (malodor and/or bacterial or other microbial contamination) in an enclosable space. The method includes introducing a magnetic impeller into a container of an apparatus and adding, into the container, a predetermined quantity of water and a formulation that releases the gaseous cleaning agent when in contact with water. The container is closed with a lid having a nozzle therein, and the enclosable space may be closed except that air circulation in the space may be maintained, to the extent necessary, with the container there inside. A magnetic field is brought into rotation in the base of the apparatus, which causes the magnetic impeller in the container, to rotate. The rotation of the magnetic impeller agitates the water and the formulation and causes an accelerated release of a gaseous cleaning agent from the formulation such that an air-borne spray that includes water droplets and gaseous cleaning agent is emitted from the nozzle of the lid. The enclosable space is maintained closed for an effective time period to treat the condition in the enclosable space.

Optionally, the method includes, the magnetic field in the base may be brought into rotation by rotating the spindle of a motor in the base of the apparatus, where the spindle of the motor is coupled to a magnet configured to create the magnetic field. Thus, the rotation of the magnet in the base creates a magnetic field of sufficient strength to rotate the magnetic impeller at a speed sufficient to create a vortex in the water.

### BRIEF DESCRIPTION OF THE DRAWINGS

The foregoing aspects and many of the attendant advantages, of the present technology will become more readily appreciated by reference to the following Detailed Description, when taken in conjunction with the accompanying simplified drawings of exemplary embodiments. The drawings, briefly described here below, are not to scale, are presented for ease of explanation and do not limit the scope of the inventions recited in the accompanying patent claims.

FIG. 1 is a schematic flow diagram of an exemplary embodiment illustrating some of the steps of the method of cleaning contaminated surfaces in an enclosed space.

FIG. 2A is an exemplary embodiment of an apparatus showing the generation of a gaseous cleaning agent from its container.

FIG. 2B is an exemplary cutaway illustration of a vehicle showing the exemplary embodiment of the apparatus of FIG. 2A emitting an air-borne gaseous cleaning agent throughout the cabin.

FIG. 3A is a schematic illustration of an exemplary embodiment of an apparatus useful in the methods and systems for cleaning contaminated surfaces in an enclosed space.

FIG. 3B is an exploded view showing components of the apparatus of FIG. 3A.

FIGS. 4A and B depict alternative views of another exemplary lid for an exemplary apparatus like that of FIGS. 3A and B.

FIG. 5 is a schematic flow diagram of an exemplary embodiment illustrating some of the steps of the method of cleaning contaminated surfaces using a timer-equipped embodiment of an apparatus according to the invention in an enclosed space.

FIG. 6A is an illustrative depiction of an exemplary embodiment of an apparatus according to the invention, equipped with a timer, that is battery-powered with the battery inside the apparatus.

FIG. 6B is an illustrative depiction of an exemplary embodiment of an inline timer for use with an apparatus according to the invention that is corded to receive power and is powered by a power source outside the apparatus.

FIG. 7 is an illustrative depiction of the use of a plurality of apparatus according to the invention to treat a plurality of enclosable spaces wherein there are surfaces that may be contaminated.

FIG. 8 is an exploded view showing components of another exemplary embodiment of an apparatus that includes a magnetic impeller and that is useful in the methods and systems for treating conditions in an enclosable space.

### DETAILED DESCRIPTION

In the following non-limiting detailed descriptions of examples of embodiments of the inventions may refer to appended Figure drawings and are not limited to the drawings, which are merely presented for enhancing explanations of features of the technology. In addition, the detailed descriptions may refer to particular terms of art, some of which are defined herein, as appropriate and necessary for clarity.

The term “cabin” as used in the specification and claims refer to a space containing contaminated surfaces that can readily be enclosed, for example by closing doors, windows and air vent system, if any, of the space such that air inside the space is neither withdrawn nor added to. The air may be allowed to re-circulate in the cabin however, by activation of an air circulation system, for example, or use of a fan in the cabin. This is useful to also treat ductwork in the air circulation system to remove malodors and to remove or reduce any microbial or viral load.

The terms “enclosable environment,” or enclosable space” is meant as synonymous with “cabin,” but for the sake of clarity these terms include, but are not limited to, rooms in a house, hotel rooms, hospital rooms, rooms in homes for the aged, intensive care units, surgery rooms, yoga rooms, gyms, restaurants, ships cabins and passenger-use spaces on cruise vessels, trains, buses, aircraft cabins, and the like. So, this should not be read as limited to commonly understood vehicular cabins. In general, the terms encompass living spaces, and other spaces, that humans use regularly, even if intermittently, and that contain surfaces on which microbial life forms can dwell as a potential source for spread of infection.

The term “coating” or “coat” as is used in reference to a coating a cleaning agent onto surfaces, means that the cleaning agent (by an air-borne mist of fine liquid droplets and a gaseous cleaning agent) contacts the surfaces, and the coating may be discontinuous in some contacted areas of surface. The surfaces may include, but are not limited to, fine surface textures, surface patterns, and tight interstitial spaces such as found, for example, in stitched seats and dash boards, couches, textiles, tiles, bedding, carpets, table tops, chairs, floors, wood, interior boundaries (such as walls, ceilings), cabinets, beds, bedding materials, etc.

The terms “contamination” or “contamination load” when used in reference to surfaces within a cabin means microbial, fungal, or viral contamination and also includes contaminants that cause a malodorous scent, for example, of decayed organic matter, fecal matter, and the like.

The term “effective period of time” as it relates to the time that the cleaning agent dwells in a cabin for cleaning of surfaces therein, the effective period may vary from about 3 to about 20 minutes; and in particular may be from about 3 to about 10 minutes. More or less time may also be effective, depending upon the degree of cleaning (extent of contaminant load reduction) to be achieved, the nature of the chemical cleaning agent used, and the number of repetitions of treatment sequentially, if such repetition is necessary. For example, some cabin spaces may have surfaces so heavily contaminated as to require more than one treatment, or to require that the gaseous cleaning agent dwell on surfaces for up to 8 hours, or overnight, to achieve a desired level of cleanliness, deodorization, sanitization or disinfection, as applicable.

The term “sealing” in the context of sealing a cabin or an enclosable environment/space means that windows doors and other ingress or exit points are closed. However, if the cabin or the enclosable environment/space has an air duct system that can be set to recirculate air, then setting it to recirculate falls within the meaning of the term “sealing” and it allows potential cleaning of the air duct system.

The term “significant reduction in contamination load” means that the contamination load of a particular contaminating species is reduced by at least 80% after cleaning in exemplary embodiments, or in some exemplary embodiments at least 98% after cleaning.

Several of the following examples discussed in relation to the Figures may relate, for ease of explanation, to vehicles, but it should be understood that the explanations are also applicable to “enclosable environments,” as defined herein.

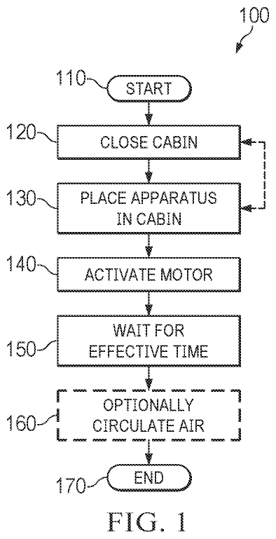

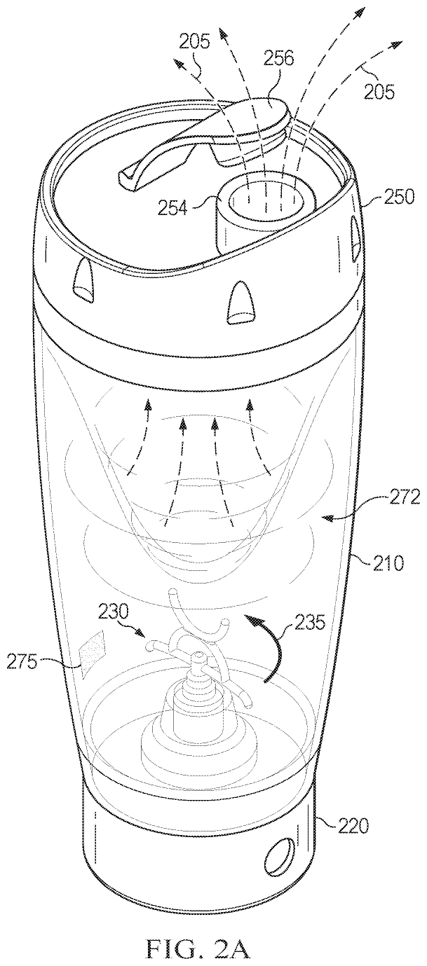

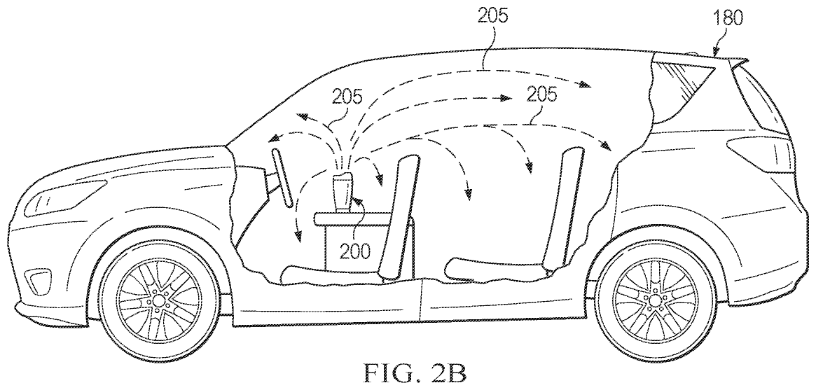

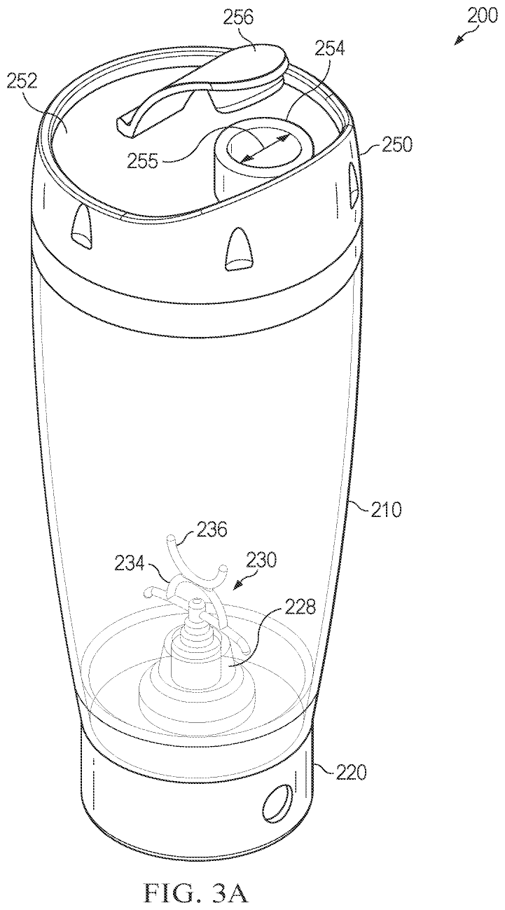

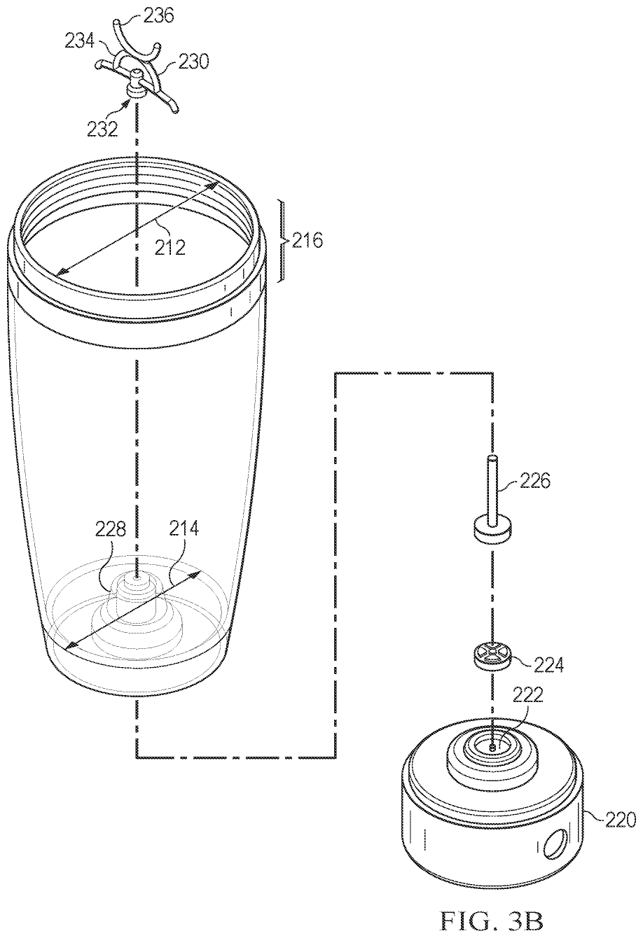

Referring to FIG. 1, an exemplary flowchart, there are several straightforward steps in the system or method depicted. Once the process starts at block 110, the cabin with contaminated surfaces inside to be treated is closed at block 120. For example, in a used car such as exemplified in FIG. 2 as 180, all doors, windows and the air circulation system, are closed off. Thus, air does not enter or leave the cabin except for natural flow around seals of doors and windows, which may occur in a closed cabin. A door is opened, and an apparatus (explained later with reference to FIGS. 3A and B, and 4A and B) containing liquid with a chemical in solid or gel pack form is placed in the cabin at block 130, and the cabin is closed. (Note that step 120 may take place after step 130; the order is not important). After a few minutes, the chemical in the closed apparatus generates a gaseous cleaning agent and the water becomes slightly cloudy as a result. At this stage the motor of the apparatus is activated in block 140. A nozzle of the apparatus is open, if it was not open already, and the nozzle is sized such that flow of gaseous agent through the nozzle erupts upward, as lava from a volcano, and flows throughout the cabin interior, as shown in FIG. 2B by arrows 205. The nozzle, as explained below, has internal structures, like baffles, that remove entrained large liquid droplets from the gaseous cleaning agent stream as it exits from the apparatus. It is theorized, without being bound, that smaller fine droplets are entrained and assist in the coating and permeation of surfaces with the gaseous cleaning agent. As with any chemical cleaning agent, the agent should be allowed to dwell on the surfaces for a period of time effective for a significant reduction in the contamination load, as in block 150. Optionally, during this period of waiting (block 150), the vehicle being treated (block 160) may activate the air circulation system, in recirculation mode. this would allow the cleaning agent to enter the ducting and filters that form part of the air circulation system and clean these of potential allergens, microbes, viruses and fungi, as well. After an effective period of time has elapsed, and entry into the cabin is deemed safe, the cabin can be opened in block 170, and the cleaning process is complete.

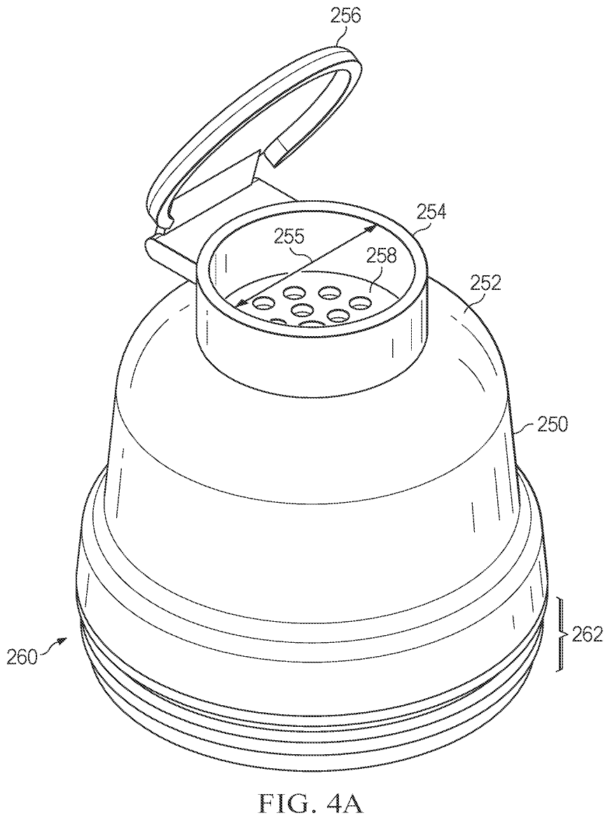

An exemplary embodiment of an apparatus useful in carrying out the systems and methods of the invention is illustrated in FIGS. 3A, 3B and 4A and 4B. As shown, the apparatus 200 has a container 210, with sides that are gently convex-curved, and that has a base 220 at one end and a lid 250 at the other end. The container 210 has a diameter 212 at its upper end that is larger than its diameter 214 near its base 220. The base 220 contains a motor driven by either a battery pack (rechargeable or not) inside the base, or by electrical connection to an electrical outlet. A spindle 226 is seated on an engaging wheel 224 that engages with motor spindle 222 and rotates in unison with motor spindle 222. An impeller 230 has a cavity 232 that friction fits to the spindle 226 so that the impeller 230 rotates as the spindle 226 rotates. The impeller in the exemplary embodiment shown has a “double horse-shoe shape” with one horse shoe 234 curved downward, and the other 236 curved upward so that the two are conjoined in a common plane at their respective apexes of curvature. This design facilitates creation of a vortex shape when liquid in container 210 is agitated by the rotating impeller in the container at speed, although other impeller shapes may also achieve the function of vortex creation. A protective cover 228 shields the motor from contents of the container 210, and fits around the spindle 226, which projects out axially through a hole in the center of the cover. The spindle is appropriately sealed against the hole to avoid or minimize leakage into a space under the cover 228.

Referring more particularly to FIGS. 4A and B, an alternative illustrated exemplary embodiment of the lid 250 can be either friction fit to the upper end of the container 210 by engaging an upper lip of the container, or can be screwed onto the container 210 by threading 260 on the lower end 262 of lid 250 that engages corresponding threading on the container upper lip (not shown). The lid 250 has a top 252 that has a nozzle 254, equipped with a nozzle closing tab 256, extending from it. Referring briefly to FIG. 2A, when in use, the exemplary container 210 is partially filled with water 272, and a solid 275 or a gel pack 275 that releases the gaseous cleaning agent when in contact with water, is placed in the water container. The cleaning gas begins to evolve more quickly when the motor is activated; gas evolution accelerates due to agitation from rotating impeller 230, as shown by arrow 235, which causes the water to form a vortex in the container 210, as shown. (In some instances it is desirable to allow the solid or gel pack to dissolve at least partially, if not completely, before the motor is activated.) Once sufficient gaseous agent has evolved, the gaseous cleaning agent erupts upward out through the nozzle as an air-borne spray to fill the cabin space and commence cleaning surfaces. Thus, the nozzle 254 has an inner diameter 255 shown in FIG. 4A that is sized to cause gaseous cleaning agent emissions from the container through the nozzle at a speed such that the emissions have both velocity and momentum to cause an air-borne spray that travels throughout the desired region of the cabin space to be cleaned. For example, the air-borne spray velocity is sufficient to travel through the cabin of a car. In other embodiments, such as for a large SUV or a cabin of a tractor trailer rig, more than one apparatus may be needed to achieve total cabin permeation by the air-borne spray. To avoid emitting foam and/or large droplets from the container, the lid includes a baffle 264, exemplified by a cart-wheel structure with spaces between the spokes covered with a fine mesh material 266, in its base area. In addition, the nozzle may include a further baffle 258, at its base, that includes perforations for flow of the gaseous cleaning agent.

While the gaseous cleaning agent has been described as chlorine dioxide, other gaseous agents that perform the same or similar function may also be useful and are encompassed in the claims here below. In addition, odorizing substances may be added to impart a pleasant smell to the interior of the cleaned cabin, or to mask any “chemical” smell.

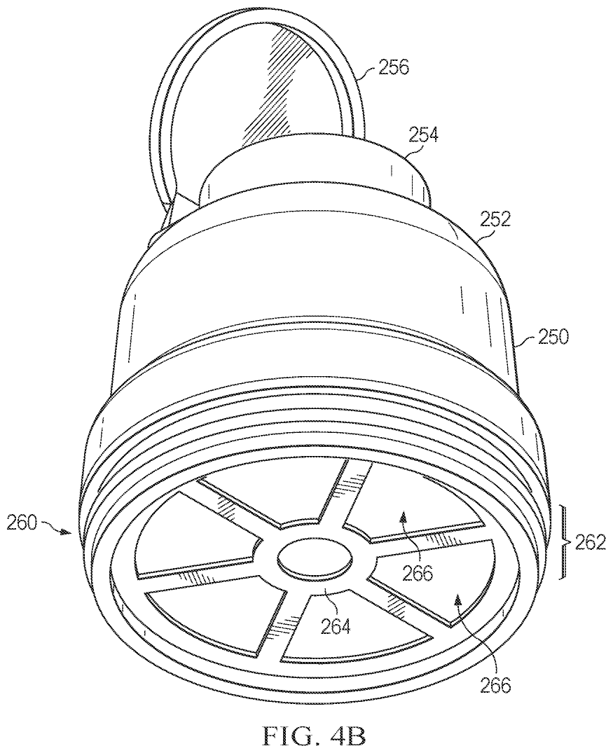

Referring to FIG. 5, an exemplary process flowchart, there are several straightforward steps in the system or method 500 depicted that uses a timer-equipped apparatus. Once the process starts at block 510, the enclosable environment with contaminated surfaces inside to be treated is closed at block 520. For example, all doors, windows and the air circulation system, are closed off so that the enclosable environment is essentially closed off from the outside environment. Thus, air does not enter or leave the enclosable environment except for any natural flow around seals of doors and windows, which may occur. Before or after this step, a door is opened, and a timer-equipped apparatus containing liquid with a chemical in solid or gel pack form is placed in the enclosable environment at block 530. (Note that step 520 may take place after step 530; the order is not important, as long as the enclosable environment is closed before the apparatus is activated.). The timer of the apparatus is set in block 540. The timer has a delay to allow an operator to exit the enclosable environment before the motor is activated. This delay can be set to a suitable time, such as 1 or minute, or more, or less, as necessary in the circumstances. After the timer activates the motor of the apparatus in step 550, generating a vortex in the container, the chemical in the apparatus generates a gaseous cleaning agent. An air-borne spray of gaseous cleaning agent and liquid exits upward, as lava from a volcano, and flows throughout the cabin interior, as shown in FIG. 2B by arrows 205. It is theorized, without being bound, that smaller fine liquid droplets are entrained and contact and assist in the coating and permeation of surfaces with the gaseous cleaning agent. The timer allows sufficient time for the cleaning agent to dwell on surfaces and clean these surfaces. Optionally, during this period the air circulation system may be shutdown or may be in recirculation mode as in block 560. If it is in recirculation mode, this would allow the cleaning agent to enter the ducting and filters that form part of the air circulation system and clean these of potential allergens, microbes, viruses and fungi, as well. After the timer expires, the motor is stopped in block 570.

After the motor is stopped, again optionally, the air circulation may be turned on to assist in dissipating the gaseous cleaning agent, as in block 580. After a period of time has elapsed, the expiration light will activate in block 590 signaling to the operator that it is safe to enter the enclosable environment because the gaseous cleaning agent has sufficiently dissipated. The period of elapsed time from motor deactivation to light activation can be set at a suitable time based on whether there is air circulation ongoing or not, and other factors that promote gaseous cleaning agent dissipation. Typically, in the range of about 3 to about 5 minutes. Then in block 595 the cleaning process is completed, and the operator can enter to retrieve the apparatus.

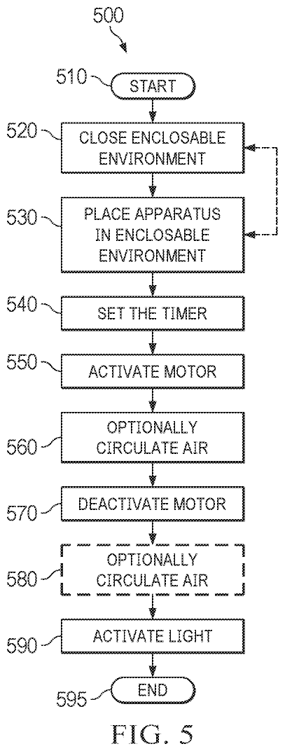

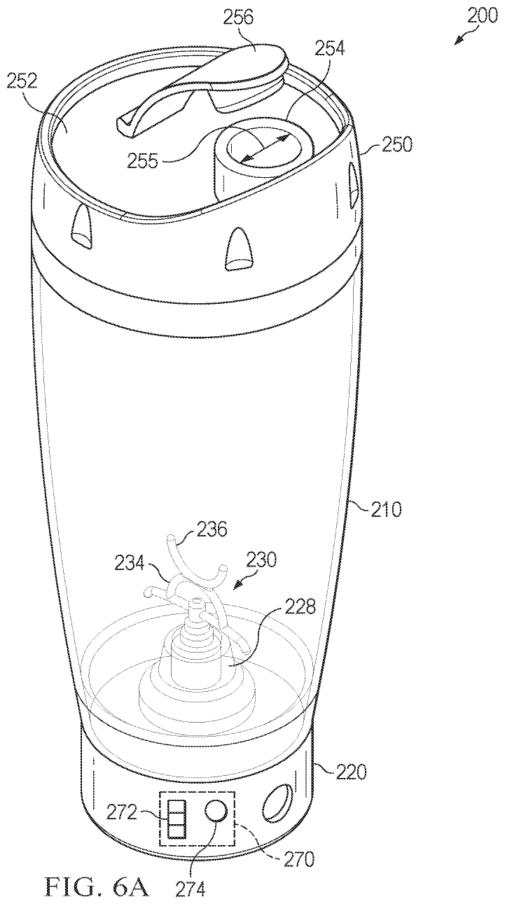

Referring now to FIG. 6A, depicting an exemplary variant of the embodiment shown in FIG. 3A, the apparatus 200 has a battery housed in the apparatus (not shown), and is equipped with a circuit board 270 having an integrated chip thereon with timer functionality. The circuit board is concealed in the base of the battery-powered apparatus, as shown, but an LED light 272 and a start button 274 both protrude from the base and are easily seen and accessed by an operator. Thus, the timer can be preset for (1) a first time that is a time delay to allow an operator time to leave after the start button 274 is depressed and (2) a second, operating time, that commences after the first time has elapsed, and when the motor (not shown) is activated. Upon elapse of the operating time, the motor is deactivated.

As an exemplary alternative, when the apparatus 200 is not battery powered, but relies on an external power source, the power can be provided, for example, by an electrical cable 288 with connector at one end 292 extending to the power source 292 and connector 290 at the other end plugging into the apparatus 200. The timer may conveniently be carried in a USB-type device 280 with integrated chip (with timer functionality) that can be connected inline in the cable 288, as shown, via opposed ends 282, 284. The USB-type IC timer device has both a start button 285 as well as an indicator light. Of course, other means may also be used, and are within the scope of this disclosure.

Further, the indicator light may be separate from the apparatus 200. Indeed, it may be independently timed. It may also be set apart from the apparatus, for example on the roof of a car being treated, or in the hallway of a hotel outside the room being treated, and the like for convenience.

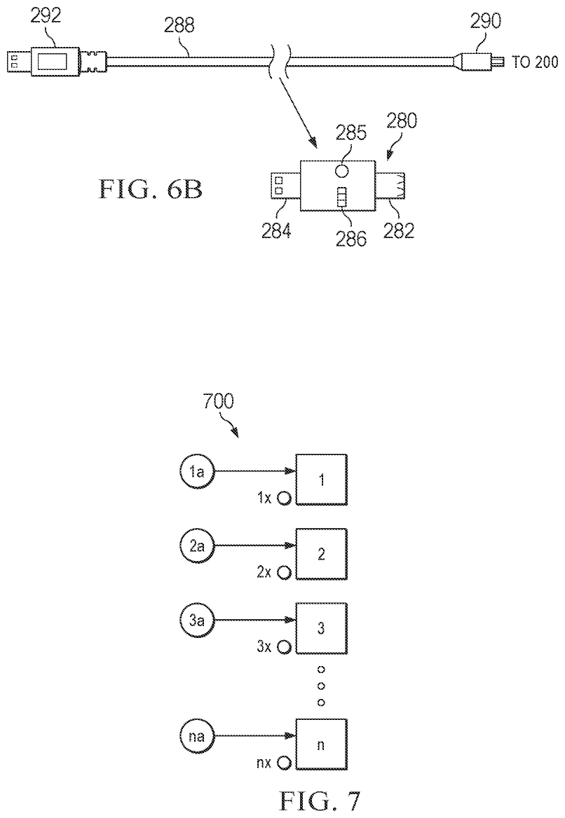

FIG. 7 depicts schematically in 700 a plurality of enclosable environments (1, 2, 3 . . . n), where n is as many enclosable environments as a lined up in the series comprising the plurality) that are each being serviced by an embodiment of an apparatus (1a, 2a, 3a . . . na) where n is as many apparatus as the enclosable environments) according to the invention. This use of a plurality n of apparatus to treat a plurality n of enclosable environments almost simultaneously, one after the other in rapid succession saves time and is an efficient use of labor. For example, in treating a number of used vehicles, these can be lined up and an operator can place an apparatus in each one after the other. Once he/she reaches the last of the plurality of vehicles n, the indicator light on the first 1x might have lighted up and the apparatus 1a in the vehicle can be retrieved. And so can all the others in series as the indicator lights (1x, 2x, 3x . . . nx) are activated. At the same time cars can be moved and others parked to take their place so that the operation becomes almost continuous.

Of course, in the example of FIG. 7, the enclosable spaces could be hotel rooms along a hallway, compartments on a train, and indeed, any enclosable environments susceptible to more efficient cleaning in series in the manner herein described.

It is recognized that certain chemical formulations may be mildly corrosive. Over a period of time, and many uses of the device described herein that mixes water with a formulation to produce a gaseous cleaning agent, it may corrode metal or electronic parts. Thus, even very minor leakage from container that generates the mixture (of cleaning agent in water) down around the spindle of the impeller into the base may cause damage in the longer term to components in the base, such as the motor or electronics. The question then is how to avoid even the most minor leakage that might be considered in-significant from passing from the container around the spindle down into the base and into contact with the motor and electricals in the base.

An alternative embodiment provides the solution: make the base a separate component that is coupled to the lower end of the container. This can be achieved, in a non-limiting example, by exterior screw thread on the base to an interior screw thread of the lower end on the container, or by some other mechanical locking means or by friction fit. The base is sealed and the inner cavity within the sealed base contains the motor there inside such that there is no fluid communication between the cavity with the interior of the container (which is a reactor-generator of the gaseous cleaning agent). Thus, leakage is avoided because there is no possible fluid communication between the interior of the container that is sealed off from the interior of the base. Thus, the sealed-in motor is effectively completely isolated from the water/formulation in the container and there is no spindle penetrating the bottom of the container to extend into the base. This is achieved by coupling the motor in the base to a magnet such that when the motor is activated, the magnet spins at a motor-controlled rate, which can be manually adjusted, in a non-limiting example, by a control knob on the outside of the base. In turn, there is a magnetic impeller placed inside the container such that the magnetic impeller responds to or interacts with the magnetic field of the rotating magnet, such that they rotate substantially in unison together. The magnetic fields of the two magnets (the motor driven one and the magnetic impeller) are sufficiently strong to swirl the water/formulation mixture in the container at a rate to cause formation of a vortex, similar to that created by a mechanical impeller explained and illustrated here above.

As with a mechanical impeller, the rotation of the magnetic impeller creates a vortex which generates an air-borne spray comprising water and gaseous cleaning agent which is expelled from the container. Moreover, the impeller's agitation of the water accelerates the rate of release of the gaseous cleaning agent into the water.

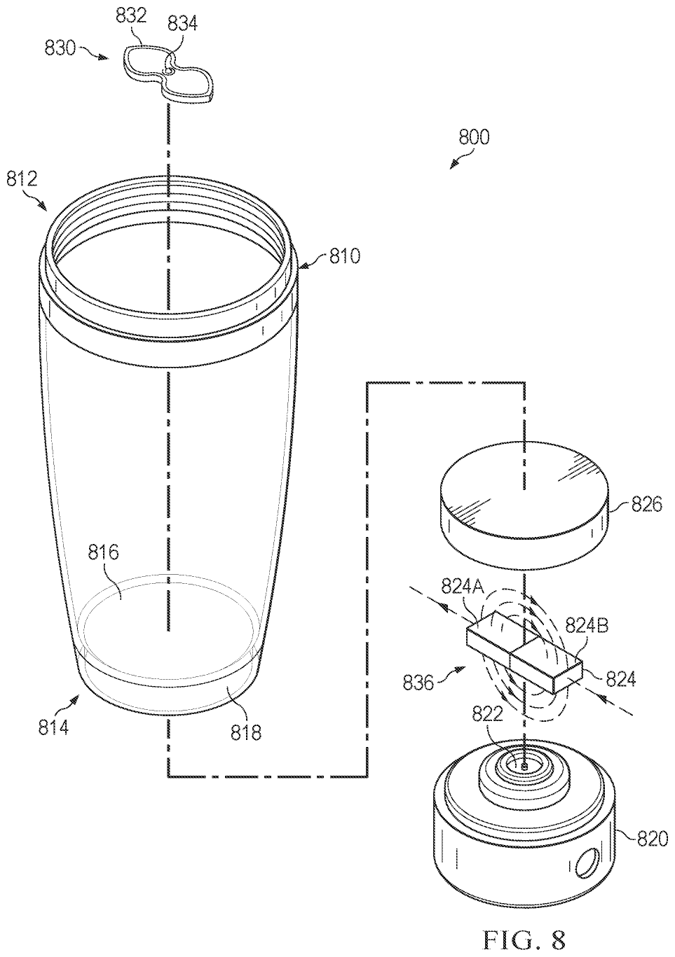

Turning to FIG. 8, this is a schematic illustration of the alternative embodiment described here above. The embodiment includes an apparatus 800 useful in carrying out the systems and methods of the invention. The apparatus 800 has a container 810, a separate base 820 that can be coupled to a lower end 814 of the container 810, and a magnetic impeller 830 that is configured for use in the container 810 to create a vortex during operation. The container 810 includes an open upper end 812 and a lower end 814. The lower end 814 is sealed by an end wall 816, thereby defining a containment space in the container 810 for containing water and the formulation, as well as receiving the magnetic impeller 830 therein. The base 820 is sealed and has a cavity that includes a motor therein (not shown). The motor is powered by either a battery pack (rechargeable or not) inside the sealed base 820, or by electrical connection to an electrical outlet. The motor housed within the sealed base 820 is rotationally coupled to driver magnet 824. In the illustrative embodiment, the driver magnet 824 is connected to the motor by spindle 822, but it may be coupled in another way, as long as the driver magnet 824 rotates as the motor (and spindle) rotates. As seen in the illustrative embodiment, driver magnet 824 can be segmented into a first portion 824A and a second portion 824B that have opposing magnetic poles. Driver magnet 824 can produce a magnetic field 836 that can be introduced into container 810. The driver magnet 824 may comprise a variety of configurations. In a non-limiting example, the driver magnet 824 may be a permanent magnet of any suitable shape/form, such as: a bar magnet, a disc magnet, a ring magnet, a rod magnet, or any other configuration that is suitable to drive the magnetic impeller. In another nonlimiting example, driver magnet 824 can be an electromagnet.

A cover (or top seal) 826 creates a water-tight seal at the upper portion of the sealed base 820. Thus, as pointed out above, the driver magnet 824 and motor are sealed off within a cavity of the base 820. The base 820 is configured to be detachably coupled to the lower end 814 of the container 810. In a non-limiting example, the cover 826 is configured to fit within the open-ended cavity 818 at the lower end of the container 810 to secure the container 810 to the base 820. The cover 826 and/or the sealed bottom portion 818 of the container 810 may be configured to form a frictional fit, or may be screwed together. Alternatively, the cover 826 may comprise a threaded surface that is configured to be threaded into a corresponding threaded surface within the sealed bottom portion 818 of the container 810.

During use, the magnetic impeller 830 is placed into the container 810, wherein the magnetic impeller is magnetically attracted to the sealed end of the container due to the magnetic attraction to the magnet in the base 820. The magnetic impeller 830 should have a shape suitable for generation of a vortex in the container during use. The magnetic impeller 830 may, in a non-limiting example, have a dumbbell shape, wherein the ends 832 of the magnetic impeller 830 have a larger size than the center of the magnetic impeller 830. This example of a dumbbell shape of the magnetic impeller 830 promotes the generation of a vortex within the container 810. In a nonlimiting example, each end 832 of the magnetic impeller 830 may include a magnet, with the faces of each end 832 having an opposite magnetic pole (e.g., left side has a north pole and the right side has a south pole). Accordingly, when the magnetic impeller 830 is placed in the container 810 coupled to the base 820, the magnetic impeller 830 will be attracted to the drive magnet 824 beneath the sealed bottom portion 818 because the ends 832 are attracted to the opposite magnetic poles of the magnet in the base 820. The magnetic impeller 830 may further include a smooth protuberance or knob 834 on one or both sides that is positioned at the center of the magnetic impeller 830. The magnetic impeller 830 spins on the knob 834, which reduces the friction with the sealed bottom portion 818 of the container 810 and allows for greater rotational velocity to generate a stronger vortex. The magnetic impeller 830 may be configured such that the length of the magnetic impeller 830 is not larger than the length (or diameter) of the container 810 at its lower end 814.

The magnetic impeller 830 may be coated with a protective polymer such as PTFE that has low friction and that will protect the magnetic impeller from the corrosive mixture in the container 810. The coating may be selected from any of the chemically resistant coatings capable of protecting the magnetic impeller from the fluids in the container.

As explained here above, the use of the magnetic impeller 830 provides an advantage of reducing or eliminating the possibility of corrosive damage to the motor in the base 820. Because there is no spindle extending from the base into the container, and the sealing off of the base from the container, the possibility of leakage from the container into the base through wear on mechanical seal(s) of the spindle is eliminated.

While examples of embodiments of the technology have been presented and described in text and some examples also by way of illustration, it will be appreciated that various changes and modifications may be made in the described technology without departing from the scope of the inventions, which are set forth in and only limited by the scope of the appended patent claims, as properly interpreted and construed.

What is claimed is:

1. An apparatus for generating and distributing a gaseous cleaning agent, the apparatus comprising:
a container having an interior volume configured to contain water and a formulation that produces the gaseous cleaning agent when in contact with the water;
a magnetic impeller configured to be placed in the container;
a base coupled to an end of the container; and
a magnetic field in the base of the apparatus, configured to be brought into rotation and to couple magnetically with the magnetic impeller, thereby bringing the magnetic impeller into rotation;
wherein, during operation of the apparatus, controlled rotation of the magnetic impeller in the container containing the water and the formulation forms a vortex in the container that accelerates release of the gaseous cleaning agent and emission of an airborne spray comprising water and the gaseous cleaning agent from the container.

2. The apparatus of claim 1, wherein:
the magnetic field is provided by a magnet in the base; and
a motor rotationally coupled to the magnet, such that rotation of a spindle of the motor causes rotation of the magnet and thereby rotation of the magnetic field.

3. The apparatus of claim 1, wherein the magnetic impeller is configured to create a vortex in the interior volume when the apparatus is in use and the magnetic impeller is rotated.

4. The apparatus of claim 1, wherein the base of the apparatus is isolated from fluid communication with the interior volume of the container.

5. The apparatus of claim 1, wherein the base comprises a mechanical interface configured to engage a corresponding interface of the container.

6. The apparatus of claim 1, the container further comprises a lid with a nozzle configured to release the gaseous cleaning agent from the container during use, wherein the lid and nozzle are configured to emit the air-borne spray of gaseous cleaning agent and entrained water through the nozzle.

7. The apparatus of claim 6, wherein the lid includes a baffle that is configured to selectively allow passage of droplets of water based on droplet size.

8. The apparatus of claim 1 further comprising a timer, the timer configured to be set for a duration of operation of the apparatus.

9. The apparatus of claim 1, further comprising external indicia that alert when an operating cycle is complete.

10. The apparatus of claim 1, wherein the apparatus comprises material resistant to release of gaseous cleaning agent.

11. An apparatus for generating and distributing a gaseous cleaning agent, the apparatus comprising:
a container having an interior configured to contain water and a formulation, the formulation producing a gaseous cleaning agent when in contact with water;
a magnetic impeller located in the container;
a base coupled to a bottom end of the container, the base having a base interior isolated from fluid communication with the interior of the container;
a motor housed within the base interior; and
a magnet housed in the base interior, the magnet rotationally coupled to the motor, the magnet having a magnetic field configured to cause the magnetic impeller to rotate when the magnet is rotated by the motor;
wherein, when the magnetic impeller is rotated, and the container contains water and a formulation, the water forms a vortex in the container.

12. The apparatus of claim 11, wherein the container further comprises a lid with a nozzle configured to release the gaseous cleaning agent from the container during use, wherein the lid and nozzle are configured to emit an air-borne spray of gaseous cleaning agent and entrained water through the nozzle.

13. The apparatus of claim 12, wherein the lid includes a baffle that is configured to selectively allow passage of droplets of water based on droplet size.

14. The apparatus of claim 11, further comprising a timer, the timer configured to be set for a duration of operation of the apparatus.

15. The apparatus of claim 11, further comprising external indicia that alert when an operating cycle is complete.

16. The apparatus of claim 11, wherein the apparatus comprises material resistant to release of the gaseous cleaning agent.

17. An apparatus for generating and distributing chlorine dioxide, the apparatus comprising:
a container resistant to chlorine dioxide corrosion having an interior configured to contain water and a formulation, the formulation producing gaseous chlorine dioxide when in contact with water;
a magnetic impeller located in the container, the magnetic impeller coated with a chlorine dioxide resistant composition;
a base coupled to a bottom end of the container, the base having a base interior isolated from fluid communication with the interior of the container;
a motor housed within the base interior; and
a magnet housed in the base interior, the magnet rotationally coupled to the motor, the magnet having a magnetic field configured to cause the magnetic impeller to rotate when the magnet is rotated by the motor;
wherein, when the magnetic impeller is rotated, and the container contains water and the formulation, the water forms a vortex in the container.

18. The apparatus of claim 17, wherein the container further comprises a lid with a nozzle configured to release the gaseous cleaning agent from the container during use, wherein the lid and nozzle are configured to emit an air-borne spray of gaseous cleaning agent and entrained water through the nozzle.

19. The apparatus of claim 17, further comprising a timer, the timer configured to be set for a duration of operation of the apparatus.

20. The apparatus of claim 17, further comprising external indicia that alert when an operating cycle is complete.
